(19) United States
(12) Patent Application Publication
(10) Pub. No.: US 2002/0157135 A1
(43) Pub. Date: Oct. 24, 2002

(54) Polynucleotide and method for selectively expressing a protein in a target cell or tissue of a plant

(75) Inventors: Ian Hector Frazer, St. Lucia (AU); Jose Ramon Botella Mesa, Kenmore (AU); Jian Zhou, Jindalee (AU); Xiao Yi Sun, Jindalee (AU)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: The University of Queensland, St. Lucia (AU)

(21) Appl. No.: 09/899,954

(22) Filed: Jul. 6, 2001

Related U.S. Application Data
(63) Continuation-in-part of application No. PCT/AU00/00007, filed on Jan. 7, 2000.

(30) Foreign Application Priority Data
Jan. 8, 1999 (AU) PP 8077/99

(51) Int. Cl. A01H 1/00; C12N 15/87

(52) U.S. Cl. 800/288; 435/455; 435/320.1

(57) ABSTRACT

A method is disclosed for constructing a synthetic polynucleotide from which a protein is selectively expressible in a target cell of a plant, relative to another cell of the plant. The method comprises selecting a first codon of a parent polynucleotide for replacement with a synonymous codon which has a higher translational efficiency in the target cell than in said other cell, and replacing said first codon with said synonymous codon to form said synthetic polynucleotide.

### CROSS-REFERENCE TO RELATED APPLICATIONS

This application is a continuation-in-part application of co-pending International Patent Application No. PCT/AU00/00007 filed Jan. 7, 2000, which designates the United States, and which claims priority of Australian Provisional Patent Application No. PP8077 filed Jan. 8, 1999.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

This invention relates generally to gene expression and in particular, to modulation of gene expression by changing codon composition of a polynucleotide. More particularly, the present invention relates to synthetic polynucleotides and methods for selectively expressing a protein in a target cell or tissue of a plant in which at least one existing codon of a parent polynucleotide encoding the protein has been replaced with a synonymous codon.

Selective targeting of genes to particular plant cells or tissues would have broad utility for producing transgenic plants with desirable novel phenotypes. However, due to a lack of tissue-specific promoters currently available, selective expression of genes in target cells or tissues of a plant has proven extremely difficult. Accordingly, there is a need to provide technologies which can facilitate selective expression of proteins to a particular target cell or tissue of a plant.

### SUMMARY OF THE INVENTION

In one aspect of the invention, there is provided a method of constructing a synthetic polynucleotide from which a protein is selectively expressible in a target cell of a plant, relative to another cell of the plant, said method comprising:

selecting a first codon of a parent polynucleotide for replacement with a synonymous codon which has a higher translational efficiency in said target cell than in said other cell; and

replacing said first codon with said synonymous codon to form said synthetic polynucleotide.

Preferably, said first codon and said synonymous codon are selected by:

comparing translational efficiencies of individual codons in said target cell relative to said other cell; and

selecting said first codon and said synonymous codon based on said measurement

A translational efficiency of a codon may be determined by any suitable technique. In a preferred embodiment, the translational efficiency of a codon is measured by:

introducing into said target cell and into said other cell, a synthetic construct comprising a reporter polynucleotide fused in frame with a tandem repeat of said individual codon, wherein said reporter polynucleotide encodes a reporter protein, and wherein said synthetic construct is operably linked to a regulatory polynucleotide; and

comparing expression of said reporter protein in said target cell and in said other cell to determine the translational efficiency of said individual codon in said target cell relative to said other cell.

Preferably, the above method is further characterized by:

introducing the synthetic construct into a progenitor cell of a cell selected from the group consisting of said target cell and said another cell; and

producing said cell from said progenitor cell, wherein said cell contains said synthetic construct.

Suitably, this method is further characterized by:

introducing the synthetic construct into a progenitor cell of a cell selected from the group consisting of said target cell and said another cell; and

growing a plant or part thereof from said progenitor cell, wherein said plant or part thereof comprises said cell containing said synthetic construct.

The above method may be further characterized by the step of introducing the synthetic construct into a plant or part thereof such that said synthetic construct is introduced into said target cell or said other cell.

Preferably, said synonymous codon corresponds to a reporter construct from which the reporter protein is expressed in said target cell at a level that is at least 110%, preferably at least 200%, more preferably at least 500%, and most preferably at least 1000%, of that expressed from the said reporter construct in said other cell.

In an alternate embodiment, the translational efficiency of a codon may be determined by measuring the abundance of an iso-tRNA corresponding to said individual codon in said target cell relative to said other cell.

Preferably, said synonymous codon corresponds to an iso-tRNA which is in higher abundance in the target cell relative to said other cell.

Preferably, selecting said first codon and said synonymous codon comprises:

measuring abundance of different iso-tRNAs in said target cell relative to said other cell; and

selecting said first codon and said synonymous codon based on said measurement, wherein said synonymous codon corresponds to an iso-tRNA which is in higher abundance in said target cell than in said other cell.

Advantageously, said synonymous codon corresponds to an iso-tRNA that is present in said target cell at a level which is at least 110%, preferably at least 200%, more preferably at least 500%, and most preferably at least 1000%, of the level that is present in said other cell.

Alternatively, the step of selecting may be characterized in that a synonymous codon according to the invention is selected from the group consisting of (1) a codon used at relatively high frequency by genes, preferably highly expressed genes, of a said target cell or tissue, (2) a codon used at relatively high frequency by genes, preferably highly expressed genes, of the plant, (3) a codon used at relatively low frequency by genes of a said one or more other cells or tissues, and (4) a codon used at relatively low frequency by genes of another organism.

The step of selecting may be characterized in that a first codon according to the invention is selected from the group consisting of (a) a codon used at relatively high frequency by genes, preferably highly expressed genes, of a said one or more other cells or tissues, (b) a codon used at relatively low frequency by genes of a said target cell or tissue, (c) a codon used at relatively low frequency by genes of the plant, and (d) a codon used at relatively high frequency by genes of another organism.

In a preferred embodiment, the method further includes the step of selecting the first codon and the synonymous codon such that said protein is expressed from said synthetic polynucleotide in said target cell or tissue at a level which is at least 110%, preferably at least 200%, more preferably at least 500%, and most preferably at least 1000%, of that expressed from said parent polynucleotide in said target cell or tissue.

Preferably, the other cell is a precursor cell of the target cell. Alternatively, the other cell may be a cell derived from the target cell.

In another aspect, the invention provides a synthetic polynucleotide constructed according to any one of the above methods.

In yet another aspect, the invention resides in a method for selectively expressing a protein in a target cell or tissue of a plant, said method comprising:

replacing a first codon of a parent polynucleotide encoding said protein with a synonymous codon to produce a synthetic polynucleotide having altered translational kinetics compared to said parent polynucleotide, such that said protein is expressed in said target cell, but such that said protein is not substantially expressed in another cell of the plant; and

introducing into a cell selected from the group consisting of said target cell and a precursor of said target cell, said synthetic polynucleotide operably linked to a regulatory polynucleotide. The protein is thereby selectively expressed in said target cell.

Preferably, said synonymous codon has a higher translational efficiency in said target cell than in said other cell.

In yet another aspect, the invention provides a method of expressing a protein in a target cell of a plant from a first polynucleotide, said method comprising:

introducing into a cell selected from the group consisting of said target cell and a precursor of said target cell, a second polynucleotide encoding an iso-tRNA, wherein said second polynucleotide is operably linked to a regulatory polynucleotide, and wherein said iso-tRNA is normally in relatively low abundance in said target cell and corresponds to a codon of said first polynucleotide. The protein is thereby expressed in the target cell.

The invention further contemplates cells or tissues containing therein the synthetic polynucleotides of the invention, or alternatively, cells or tissues produced from the methods of the invention.

In yet another aspect, the invention resides in a transgenic plant or plant part containing cells or tissues having the synthetic polynucleotides of the invention.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

In the drawings:

FIG. 1 is a general map of the vectors described in Example 6. The vector pSLJ75515 was used to introduce several cassettes each containing the 35S CaMV promoter, a specific multiple cloning site (MCS) and nos terminator. pSLJ75515 is a derivative of pRK290 (Jones et al., 1992, Transgen Res. 1:285-297). The total size of all the binary vectors described herein is 28kb.

FIG. 2 is a schematic representation of the construction of the different sense and antisense cassettes described in Example 6. The most prominent details of the PRL2 library vector and the different cassettes constructed to insert EST clones in either sense or antisense orientation under the control of the CaMV 35S promoter and nos terminator. Restriction enzyme sites that can be used for the cloning of EST inserts are shown. LB and RB represent T-DNA left and right border sequences respectively. The nopaline synthase promoter and terminators are represented as nos and nos3′, respectively. 35S and ocs 3′ indicates CaMV 35S promoter and octopine synthase 3′ end repsectively. BAR indicates the bialophos resistance gene, which encodes the enzyme phosphinotricin acetyltransferase (Thompson et al., 1987, EMBO J. 6:2519-2524).

### DETAILED DESCRIPTION OF THE INVENTION

The present invention is based, at least in part, on the discovery in copending International Application No. PCT/AU98/00530 that the intracellular abundance of different species of isoaccepting transfer RNAs (iso-tRNAs) in different cell or tissue types contributes to the expression of messenger RNA (mRNA) by determining the rate of protein translation from the mRNA. By altering the structure of a protein-encoding polynucleotide to replace existing codons with synonymous codons that correspond to iso-tRNAs that are in higher or lower abundance intracellularly, it was shown that the translational efficiency of a mRNA can be dramatically modulated both in vitro and in vivo. This ability to control the translational kinetics of a modified mRNA allows for the modified mRNA to be selectively expressible in a particular cell or tissue, or alternatively in a cell or tissue in a specific state of differentiation or in a specific stage of the cell cycle.

Although the subject matter of PCT/AU98/00530 is primarily concerned with selective expression of a protein in a target cell or tissue of a mammal, the present inventors believe that different species of iso-tRNAs would be differentially expressed in different cells or tissues of most multi-cellular organism. Put another way, the inventors consider that the translational efficiencies of different codons will vary in different cells or tissues. Such differences can be exploited together with codon composition of a gene to regulate and direct expression of a protein to a particular cell or cell type of a plant, including cells in a selected tissue. Alternatively, these differences can be exploited together with codon composition of a gene to regulate and direct expression of a protein to a cell or tissue of a plant in a selected state of differentiation or in a selected stage of the cell cycle.

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “expressible” is meant expression of a protein to a level sufficient to effect a particular function associated with the protein. By contrast, the terms “not expressible” and “not substantially expressible” as used interchangeably herein refers to (a) no expression of a protein, (b) expression of a protein to a level that is not sufficient to effect a particular function associated with the protein, (c) expression of a protein, which cannot be detected by a monoclonal antibody specific for the protein, or (d) expression of a protein, which is less that 1% of the level expressed in a wild-type cell that normally expresses the protein.

By “expressing said synthetic construct” is meant transcribing the synthetic construct such that mRNA is produced.

By “expression vector” is meant any autonomous genetic element capable of directing the synthesis of a protein encoded by the vector. Such expression vectors are known by practitioners in the art.

As used herein, the term “function” refers to a biological, enzymatic, physical, chemical or therapeutic function.

The terms “growing” or “regeneration” as used herein mean growing a whole, differentiated plant from a plant cell, a group of plant cells, a plant part (including seeds), or a plant piece (e.g., from a protoplast, callus, or tissue part).

By “highly expressed genes” is meant genes that express high levels of mRNA, and preferably high level of protein, relative to other genes.

By “isoaccepting transfer RNA” or “iso-tRNA” is meant one or more transfer RNA molecules that differ in their anticodon nucleotide sequence but are specific for the same amino acid.

By “marker gene” is meant a gene that imparts a distinct phenotype to cells expressing the marker gene and thus allow such transformed cells to be distinguished from cells that do not have the marker. A selectable marker gene confers a trait for which one can ‘select’ based on resistance to a selective agent (e.g., a herbicide, antibiotic, radiation, heat, or other treatment damaging to untransformed cells). A screenable marker gene (or reporter gene) confers a trait that one can identify through observation or testing, i.e., by ‘screening’ (e.g. β-glucuronidase, luciferase, or other enzyme activity not present in untransformed cells).

By “natural gene” is meant a gene that naturally encodes the protein. However, it is possible that the parent polynucleotide encodes a protein that is not naturally-occurring but has been engineered using recombinant techniques.

The term “non-cycling cell” as used herein refers to a cell that has withdrawn from the cell cycle and has entered the GO state. In this state, it is known that transcription of endogenous genes and protein translation are at substantially reduced levels compared to phases of the cell cycle, namely G1, S, G2 and M. By contrast, the term “cycling cell” as used herein refers to a cell, which is in one of the above phases of the cell cycle.

By “obtained from” is meant that a sample such as, for example, a polynucleotide extract or polypeptide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “operably linked” is meant that transcriptional and translational regulatory polynucleotides are positioned relative to a polypeptide-encoding polynucleotide in such a manner that the polynucleotide is transcribed and the polypeptide is translated.

As used herein, “plant” and “differentiated plant” refer to a whole plant or plant part containing differentiated plant cell types, tissues and/or organ systems. Plantlets and seeds are also included within the meaning of the foregoing terms. Plants included in the invention are any plants amenable to transformation techniques, including angiosperms, gymnosperms, monocotyledons and dicotyledons.

The term “plant cell” as used herein refers to protoplasts, gamete-producing cells, and cells which regenerate into whole plants. Plant cells include cells in plants as well as protoplasts in culture.

By “plant tissue” is meant differentiated and undifferentiated tissue derived from roots, shoots, pollen, seeds, tumor tissue, such as crown galls, and various forms of aggregations of plant cells in culture, such as embryos and calluses.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polynucleotide” or “nucleic acid” as used herein designates mRNA, RNA, cRNA, cDNA or DNA. The term typically refers to oligonucleotides greater than 30 nucleotide residues in length.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labeled directly or indirectly.

The terms “precursor cell or tissue” and “progenitor cell or tissue” as used herein refer to a cell or tissue that can gives rise to a particular cell or tissue in which protein expression is to be targeted or in which translational efficiency of a codon is to be determined.

By “recombinant polypeptide” is meant a polypeptide made using recombinant techniques, i.e., through the expression of a recombinant or synthetic polynucleotide.

“Stringency” as used herein, refers to the temperature and ionic strength conditions, and presence or absence of certain organic solvents, during hybridization. The higher the stringency, the higher will be the degree of complementarity between immobilized polynucleotides and the labeled polynucleotide.

“Stringent conditions” refers to temperature and ionic conditions under which only polynucleotides having a high frequency of complementary bases will hybridize. The stringency required is nucleotide sequence dependent and depends upon the various components present during hybridization. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridizes to a complementary probe.

The term “synthetic polynucleotide” as used herein refers to a polynucleotide formed in vitro by the manipulation of a polynucleotide into a form not normally found in nature. For example, the synthetic polynucleotide can be in the form of an expression vector. Generally, such expression vectors include transcriptional and translational regulatory polynucleotide operably linked to the polynucleotide.

The term “synonymous codon” as used herein refers to a codon having a different nucleotide sequence than another codon but encoding the same amino acid as that other codon.

By “translational efficiency” is meant the efficiency of a cell's protein synthesis machinery to incorporate the amino acid encoded by a codon into a nascent polypeptide chain. This efficiency can be evidenced, for example, by the rate at which the cell is able to synthesize the polypeptide from an RNA template comprising the codon, or by the amount of the polypeptide synthesized from such a template.

The term “transformation” means alteration of the genotype of a host plant by the introduction of a chimeric nucleic acid.

By “transgenote” is meant an immediate product of a transformation process.

By “vector” is meant a polynucleotide molecule, preferably a DNA molecule derived, for example, from a plasmid, bacteriophage, or plant virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

2. Selection of Synonymous Codons

According to the present invention, selective targeting of protein expression to a plant cell is effected by replacing at least one existing codon (sometimes referred to as a “first codon”) of a parent polynucleotide encoding the protein with a synonymous codon (i.e. one which encodes the same amino acid residue as the first codon).

Replacement of synonymous codons for existing codons is not new per se. In this regard, we refer to International Application Publication No WO 96/09378 which utilizes such substitution to provide a method of expressing proteins of eukaryotic and viral origin at high levels in in vitro mammalian cell culture systems, the main thrust of that method being the harvesting of such proteins. In distinct contrast, the present invention utilizes substitution of one or more codons in a gene for targeting expression of the gene to particular cells or tissues with the ultimate aim of producing transgenic plants with novel phenotypes.

The present method preferably includes the step of selecting the codons such that the synonymous codon has a higher translational efficiency in said target cell or tissue (“cell or tissue” is sometimes referred to herein as “cell/tissue”) relative to said one or more other cells or tissues. As used herein, expression of a protein in a tissue refers alternatively to expression of the protein within a cell of the tissue or production of the protein within a cell and export of the protein from the cell to, for example, the extracellular matrix of a tissue.

Methods for determining translational efficiencies of different codons in and between different cells or tissues are described in detail in Section 3. The translational efficiencies so determined can be used to identify which isocoding triplets are differentially translated between the different cells or tissues. In a typical scenario, there will be: (A) codons with higher translational efficiencies in a target cell/tissue relative to one or more other cells/tissues; (B) codons with higher translational efficiencies in the one or more other cells/tissues relative to the target cell/tissue; and (C) codons with about the same translational efficiencies in the target cell/tissue relative to the one or more other cells/tissues. Synonymous codons are selected such that they correspond to (A) codons. Preferably, a synonymous codon is selected such that it has the largest difference in translational efficiency in the target cell or tissue relative to the existing codon that it replaces. Existing codons in a parent polynucleotide are preferably selected such that they do not have the same translational bias as the synonymous codons with respect to the target cell/tissue and the one or more other cell/tissue (i.e., existing codons should preferably not correspond to (A) codons). However, existing codons can have similar translational efficiencies in each of the target cell/tissue and the one or more other cells/tissues (i.e., existing codons can correspond to (C) codons. They can also have a translational bias opposite to that of the synonymous codons (i.e., existing codons can, and preferably do, correspond to (B) codons).

Suitably, a synonymous codon has a translational efficiency in the target cell/tissue that is at least 110%, preferably at least 200%, more preferably at least 500%, and still more preferably at least 1000%, of that in the other cell(s)/tissue(s). In the case of two or more synonymous codons having similar translational efficiencies in the target cell/tissue relative to the other cell(s)/tissue(s), it will be appreciated that any one of these codons can be used to replace the existing codon.

It is preferable but not necessary to replace all the existing codons of the parent polynucleotide with synonymous codons having higher translational efficiencies in the target cell/tissue compared to the other cells/tissues. Increased expression can be accomplished even with partial replacement Suitably, the replacement step affects 5%, 10%, 15%, 20%, 25%, 30%, more preferably 35%, 40%, 50%, 60%, 70% or more of the existing codons of the parent polynucleotide.

The difference in level of protein expressed in the target cell/tissue from a synthetic polynucleotide relative to that expressed in the other cell(s)/tissue(s) depends on the percentage of existing codons replaced by synonymous codons, and the difference in translational efficiencies of the synonymous codons in the target cell/tissue relative to the other cell(s)/tissue(s). Put another way, the fewer such replacements, and/or the smaller the difference in translational efficiencies of the synonymous between the different cells/tissues, the smaller the difference in protein expression between the target cell/tissue and the other cell(s)/tissue(s) will be. Conversely, the more such replacements, and/or the greater the difference in translational efficiencies of the synonymous between the different cells/tissues, the greater the difference in protein expression between the target cell/tissue and the other cell(s)/tissue(s) will be. The inventors have found in this respect that a protein can be expressed from a synthetic polynucleotide in a target cell/tissue at levels greater than 10,000-fold over those expressed in another cell/tissue.

In contrast to differential protein expression between different cells/tissues, it will be appreciated that a synthetic polynucleotide may be tailored with synonymous codons such that expression of a protein in a target cell is enhanced. In this regard, the difference in level of protein expressed in the target cell/tissue from a synthetic polynucleotide relative to that expressed from a parent polynucleotide depends on the percentage of existing codons replaced by synonymous codons, and the difference in translational efficiencies between the existing codons and the synonymous codons in the target cell/tissue. Put another way, the fewer such replacements, and/or the smaller the difference in translational efficiencies between the synonymous and existing codons, the smaller the difference in protein expression between the synthetic polynucleotide and parent polynucleotide will be. Conversely, the more such replacements, and/or the greater the difference in translational efficiencies between the synonymous and existing codons, the greater the difference in protein expression between the synthetic polynucleotide and parent polynucleotide will be. The inventors have found in this respect that a protein can be expressed from a synthetic polynucleotide in a target cell/tissue at levels greater than 10,000-fold than from a parent polynucleotide.

Preferably, the at least one existing codon and the synonymous codon are selected such that said protein is expressed from said synthetic polynucleotide in said target cell or tissue at a level which is at least 110%, preferably at least 200%, more preferably at least 500%, and most preferably at least 1000%, of that expressed from said parent polynucleotide in said target cell or tissue.

In a preferred embodiment, the synonymous codon is a codon which has a higher translational efficiency in the target cell or tissue relative to a precursor cell or tissue of the target cell or tissue.

In an alternate embodiment, the synonymous codon is a codon which has a higher translational efficiency in the target cell or tissue relative to a cell or tissue derived from said target cell or tissue.

The two codons can be selected by measuring translational efficiencies of different codons in the target cell or tissue relative to the one or more other cells or tissues and identifying the at least one existing codon and the synonymous codon based on this measurement.

3. Methods of Determining Codon Translational Efficiency

3.1. Expressing a Synthetic Construct Comprising a Tandem Repeat of Identical Codons Fused in Frame to a Reporter Polynucleotide

A major aspect of the present invention is based, at least in part, on the discovery that different but synonymous stretches of identical codons fused respectively in frame with a reporter polynucleotide can give rise to different levels of reporter protein expressed within a given cell type. Not wishing to be bound by any particular theory, it is believed that a tandem series of identical codons causes a ribosome to pause during translation if the iso-tRNA corresponding to the identical codons is limiting. In this regard, it is known that ribosomal pausing leads to a failure to complete a nascent polypeptide chain and an uncoupling of transcription and translation. Accordingly, the levels of reporter protein expressed in the different cells or tissues are sensitive to the intracellular abundance of the iso-tRNA species corresponding to the identical codons and therefore provide a direct correlation of a cell's or tissue's preference for translating a given codon. This means, for example, that if the levels of the reporter protein obtained in a cell or tissue type to which a synthetic construct having a tandem series of identical first codons is provided are lower than the levels expressed in the same cell or tissue type to which a different synthetic construct having a tandem series of identical second codons is provided (i.e., wherein the first codons are different from, but synonymous with, the second codons), then it can be deduced that the cell or tissue has a higher preference for the second codon relative to the first codon with respect to translation. Put another way, the second codon has a higher translational efficiency compared to the first codon in the cell or tissue type.

With regard to differential protein expression between different cell or tissue types, it will be appreciated that if the levels of the reporter protein obtained in a target cell or tissue type to which a synthetic construct having a tandem series of identical codons is provided are lower than the levels expressed in another cell or tissue type to which the same synthetic construct is provided, then it can be deduced that the target cell or tissue has a higher preference for the codon relative to the other cell or tissue with respect to translation. Put another way, the codon has a higher translational efficiency in the target cell or tissue compared to the other cell or tissue type.

Suitably, the tandem repeat comprises at least three identical codons. Preferably, the tandem repeat comprises four identical codons, more preferably five or seven identical codons and most preferably six identical codons.

The tandem repeat can be fused at a location adjacent to, or within, the reporter polynucleotide. The location is preferably selected such that the tandem repeat interferes with translation of at least a detectable portion of the reporter protein such that expression of the protein can be detected or assessed. Preferably, the tandem repeat is located immediately upstream (translationally) from the reporter polynucleotide.

It is of course possible that a tandem repeat of identical amino acid residues (e.g., an oligo-proline repeat) can render the reporter protein unstable. Typically, protein instability is detected when expression of the reporter gene is not detectable with any choice of isoaccepting codon specific for the amino acid corresponding to the tandem repeat. The inventors have found in this regard that protein instability can be alleviated by use of at least one spacer codon within the tandem repeat of identical codons, wherein the spacer codon encodes a neutral amino acid.

The at least one spacer codon can be placed adjacent to, or interposed between, some or all of the identical codons corresponding to the tandem repeat. For example, a suitable interposition for a penta-repeat of identical codons can be selected from the group consisting of: (a) I-S-I-S-I-S-I-S-I-S; (b) S-I-S-I-S-I-S-I-S-I; (c) I-S-I-S-I-I-S-I; (d) I-S-I-I-S-I-S-I; (e) I-S-I-S-I-I-I; (f) I-I-S-I-S-I-I; (g) I-I-I-S-I-S-I; (h) I-S-I-I-S-I-I; (i) I-I-S-I-I-S-I; (j) I-S-I-I-I-S-I; (k) I-S-I-I-I-I; (l) I-I-S-I-I-I; (m) I-I-I-S-I-I; and (n) I-I-I-I-S-I, wherein I corresponds to an identical codon of a tandem repeat and S corresponds to a spacer codon.

Preferably, a spacer codon is efficiently translated in the cell or tissue type relative to other synonymous codons. This is important so that translation of the spacer codon is not rate limiting. The neutral amino acid includes, but is not restricted to, alanine and glycine.

The reporter polynucleotide can encode any suitable protein for which expression can be detected directly or indirectly such as by suitable assay. Suitable reporter polynucleotides include, but are not restricted to, polynucleotides encoding β-galactosidase, firefly luciferase, alkaline phosphatase, chloramphenicol acetyltransferase (CAT), β-glucuronidase (GUS), herbicide resistance genes such as the bialophos resistance (BAR) gene that confers resistance to the herbicide BASTA, and green fluorescent protein (GFP). Assays for the activities associated with such proteins are known by those of skill in the art. Preferably, the reporter polynucleotide encodes GFP.

Persons of skill in the art will appreciate that reporter polynucleotides need not correspond to a full-length gene encoding a particular reporter protein. In this regard, the invention also contemplates reporter polynucleotide sub-sequences encoding desired portions of a parent reporter protein, wherein an activity or function of the parent protein is retained in said portions. A polynucleotide sub-sequence encodes a domain of the reporter protein having an activity associated therewith and preferably encodes at least 10, 20, 50, 100, 150, or 500 contiguous amino acid residues of the reporter protein.

The instant method is applicable to any suitable plant cell or tissue type. The cell or tissue type can be of any suitable lineage.

Suitable methods for isolating particular cells or tissues are known to those of skill in the art. For example, one can take advantage of one or more particular characteristics of a cell or tissue to specifically isolate the cell or tissue from a heterogeneous population. Such characteristics include, but are not limited to, anatomical location of a tissue, cell density, cell size, cell morphology, cellular metabolic activity, cell uptake of ions such as Ca²⁺, K⁺, and H⁺ ions, cell uptake of compounds such as stains, markers expressed on the cell surface, protein fluorescence, and membrane potential. Suitable methods that can be used in this regard include surgical removal of tissue, flow cytometry techniques such as fluorescence-activated cell sorting (FACS), immunoaffinity separation (e.g., magnetic bead separation such as Dynabead™ separation), density separation (e.g., metrizamide, Percoll™, or Ficoll™ gradient centrifugation), and cell-type specific density separation.

In an alternate embodiment, progenitor cells or tissues can be used for initially introducing the synthetic construct. Any suitable progenitor cell or tissue can be used which gives rise to a particular plant cell or tissue of interest for which codon preference is to be ascertained. For example, a suitable progenitor of a plant includes, but is not restricted to, a meristematic cell and a callus tissue, respectively.

In another embodiment, the synthetic construct can be introduced first into a plant or part thereof before subsequent expression of the construct in a particular cell or tissue type of the plant.

The invention further provides for the analysis of codon translational efficiencies in a plant cell a synthetic construct comprising a reporter polynucleotide fused in frame with a tandem repeat of (e.g., 2, 3, 4, 5, 6, or 7 or more) identical codons, wherein said reporter polynucleotide encodes a reporter protein, and wherein said synthetic construct is operably linked to one or more regulatory polynucleotides.

The construction of the synthetic construct can be effected by any suitable technique. For example, in vitro mutagenesis methods can be employed, which are known to those of skill in the art. Suitable mutagenesis methods are described for example in the relevant sections of Ausubel, et al. (supra) and of Sambrook, et al., (supra) which are incorporated herein by reference. Alternatively, suitable methods for altering DNA are set forth, for example, in U.S. Pat. Nos. 4,184,917, 4,321,365 and 4,351,901, which are incorporated herein by reference. Instead of in vitro mutagenesis, the synthetic polynucleotide can be synthesized de novo using readily available machinery. Sequential synthesis of DNA is described, for example, in U.S. Pat. No 4,293,652, which is incorporated herein by reference. However, it should be noted that the present invention is not dependent on, and not directed to, any one particular technique for constructing the synthetic construct.

Regulatory polynucleotides which can be utilized to regulate expression of the synthetic polynucleotide include, but are not limited to, a promoter, an enhancer, and a transcription terminator. Such regulatory polynucleotides are known to those of skill in the art. The construct preferably comprises at least one promoter.

Regulatory polynucleotides which can be utilized to regulate expression of the synthetic construct include, but are not limited to, a promoter, an enhancer, and a transcriptional terminator. Such regulatory polynucleotides are known to those of skill in the art. Suitable promoters that can be utilized to induce expression of the polynucleotides of the invention include constitutive promoters and inducible promoters.

3.2. Determination of Abundance of Different

tRNA Species in and/or Between Different Cells of a Plant

The present invention contemplates any suitable method for determining the abundance of different iso-tRNA species in and/or between different cell or tissue types of a plant. For example, such method can include isolating a particular cell or tissue from a plant, preparing an RNA extract from the cell or tissue which extract includes tRNA, and probing the extract with polynucleotides having different nucleic acid sequences, each being specific for a particular iso-tRNA to thereby determine the relative abundance of different iso-tRNAs in said cell or tissue. Preferably, this method is applied to two or more different cell or tissue types of the plant to determine the relative abundance of different iso-tRNAs between those cell or tissue types.

Suitable methods for isolating particular cells or tissues are known to those of skill in the art and are described, for example, in Section 3.1 above.

Any suitable method for isolating total RNA from a plant cell or tissue can be used. Typical procedures contemplated by the invention are described in CURRENT PROTOCOLS IN MOLECULAR BIOLOGY (Ausubel, et al., Eds.) (John Wiley & Sons, Inc. 1997), hereby incorporated by reference, at page 4.2.1 through page 4.2.7. Preferably, techniques which favor isolation of tRNA are employed as, for example, described in Brunngraber, E. F. (1962, Biochem. Biophys. Res. Commun. 8: 1-3), which is hereby incorporated by reference.

The probing of an RNA extract is suitably effected with different oligonucleotide sequences each being specific for a particular iso-tRNA. Of course it will be appreciated that for a given plant, oligonucleotide sequences would need to be selected which hybridize specifically with particular iso-tRNA sequences expressed by the plant. Such selection is within the realm of one of ordinary skill in the art based on any known iso-tRNA sequence. Reference can be made in this regard to a compilation of tRNA sequences and sequences of tRNA genes described in Sprinzl et al (1996, Nucleic Acids Res. 24(1): 68-72; 1998, 26(1): 148-53; Dec. 20, 1999 http://www.uni-bayreuth.de/departments/biochemie/trna/), the entire disclosures of which are incorporated herein by reference. For example, in the case of Arabidopsis thaliana, iso-tRNA sequences from which oligonucleotide sequences may be selected include, but are not limited to, those listed in TABLE 1 which sequences are incorporated herein by reference.

TABLE 1
Nucleic acid sequences of various
iso-tRNA species expressed by Arabidopsis thaliana

| | | | | | Codon |
|---|---|---|---|---|---|
| GenBank | Amino | Codon | GenBank | Amino | Recog- |
| Accession # | Acid | Recognized | Accession # | Acid | nized |
| AB005780 | Asp | GAC | D50933 | Met | ATG |
| AB005781 | Asp | GAC | D50934 | Met | ATG |
| AB005779 | Cys | TGC | M58320 | Ser | TGA |
| D17336 | Cys | TGC | L34745 | Trp | TGG |
| D50935 | Gln | CAA | L35907 | Trp | TGG |
| AB005786 | Glu | GAG | L35908 | Trp | TGG |
| AB005782 | Gly | GGC | L35909 | Trp | TGG |
| AB005783 | Gly | GGC | M35957 | Tyr | AAT |
| AB005784 | Gly | GGC | M35958 | Tyr | TCA |
| AB005785 | Gly | GGC | X54513 | Val | GUU |

Typically, the abundance of iso-tRNA species can be determined by blotting techniques that include a step whereby a sample RNA or tRNA extract is immobilized on a matrix (preferably a synthetic membrane such as nitrocellulose), a hybridization step, and a detection step. Northern blotting can be used to identify an RNA sequence that is complementary to a polynucleotide probe. Alternatively, dot blotting and slot blotting can be used to identify complementary DNA/RNA or RNA/RNA nucleic acid sequences. Such techniques are known by those skilled in the art, and have been described, for example, in Ausubel, et al (supra) at pages 2.9.1 through 2.9.20.

According to such methods, a sample of tRNA immobilized on a matrix is hybridized under stringent conditions to a complementary polynucleotide (such as one having a sequence mentioned above) which is labeled, for example, radioactively, enzymatically or fluorochromatically.

While stringent washes are typically carried out at temperatures from about 42° C. to 68° C., one skilled in the art will appreciate that other temperatures can be suitable for stringent conditions. Maximum hybridization typically occurs at about 20° to 25° below the Tₘ for formation of a DNA-DNA hybrid. It is known in the art that the Tₘ is the melting temperature, or temperature at which two complementary polynucleotides dissociate. Methods for estimating Tₘ are known in the art (see, e.g., CURRENT PROTOCOLS IN MOLECULAR BIOLOGY supra at page 2.10.8). Maximum hybridization typically occurs at about 10° to 15° below the Tₘ for a DNA-RNA hybrid.

Other stringent conditions are known in the art. A skilled artisan will recognize that various factors can be manipulated to optimize the specificity of the hybridization. Optimization of the stringency of the final washes can serve to ensure a high degree of hybridization.

Methods for detecting labeled polynucleotides hybridized to an immobilized polynucleotide are known to practitioners in the art. Such methods include autoradiography, chemiluminescent, fluorescent and calorimetric detection.

Advantageously, the relative abundance of an iso-tRNA between two or more plant cells or tissues can be determined by comparing the respective levels of binding of a labeled polynucleotide specific for the iso-tRNA to equivalent amounts of immobilized RNA obtained from the two or more cells or tissues. Similar comparisons are suitably carried out to determine the respective relative abundance of other iso-tRNAs between the two or more cells or tissues. One of ordinary skill in the art will thereby be able to determine a relative tRNA abundance table (see for example TABLE 2 of International Application No. PCT/AU98/00530 the entire contents of which are hereby incorporated by reference) for different cells or tissues of a plant. From such comparisons, one or more synonymous codons can be selected such that the or each synonymous codon corresponds to an iso-tRNA which is in higher abundance in the target cell or tissue relative to other cells or tissues of the plant.

In the present embodiment, a synonymous codon is preferably selected such that its corresponding iso-tRNA is present in the target cell or tissue at a level which is at least 110%, preferably at least 200%, more preferably at least 500%, and most preferably at least 1000%, of that present in one or more other cells or tissues of the plant.

3.3. Analysis of Codon Usage

Alternatively, synonymous codons can be selected by analyzing the frequency at which codons are used by genes expressed in (i) particular cells or tissues of a plant, (ii) substantially all cells or tissues of the plant, or (iii) an organism which can infect particular cells or tissues of the plant.

Codon frequency tables as well as suitable methods for determining frequency of codon usage in an organism are described, for example, in an article by Sharp et al (1988, Nucleic Acids Res. 16 8207-8211), which is incorporated herein by reference. Reference also can be made to an article by Nakamura et al (2000, Nucleic Acids Res 28(1): 292, incorporated herein by reference), which presents the frequency of each of the 257 468 complete protein-coding sequences (CDSs) compiled from the taxonomic divisions of the GenBank DNA sequence database. The sum of the codons used by 8792 organisms is also calculated. The data files relating to this article can be obtained from the anonymous ftp sites of DDBJ, Kazusa and EBI. A list of the codon usage of genes and the sum of the codons used by each organism can, for example, be obtained through the web site http://www.kazusa.orjp/ codon/.

The relative level of gene expression (e.g., detectable protein expression vs. no substantial or detectable protein expression) can provide an indirect measure of the relative translational efficiencies of codons, the relative abundance of specific iso-tRNAs expressed, or both, in different cells or tissues. For example, a virus can be capable of propagating within a first cell or tissue (which can include a cell or tissue at a specific stage of differentiation) but can be substantially incapable of propagating in a second cell or tissue (which can include a cell or tissue at another stage of differentiation). Comparison of the pattern of codon usage by genes of the virus with the pattern of codon usage by genes expressed in the second cell or tissue can thus provide indirectly a set of codons that have high translational efficiencies and a set of codons that have low translational efficiencies in the first cell or tissue relative to the second cell or tissue. Simultaneously, the above comparison can also provide indirectly a set of codons that that have higher translational efficiencies and a set of codons that have low translational efficiencies in the second cell or tissue relative to the first cell or tissue.

From the foregoing, a synonymous codon according to the invention can correspond to a codon selected from the group consisting of (1) a codon used at relatively high frequency by genes, preferably highly expressed genes, of a target cell or tissue, (2) a codon used at relatively high frequency by genes, preferably highly expressed genes, of the plant, (3) a codon used at relatively low frequency by genes of one or more other cells or tissues, and (4) a codon used at relatively low frequency by genes of another organism.

By contrast, an existing codon according to the invention can correspond to a codon selected from the group consisting of (a) a codon used at relatively high frequency by genes, preferably highly expressed genes, of one or more other cells or tissues, (b) a codon used at relatively low frequency by genes of a target cell or tissue, (c) a codon used at relatively low frequency by genes of the plant, and (d) a codon used at relatively high frequency by genes of another organism.

Preferably, the genes from which codon frequency data are obtained do not relate to mitochondrial genes.

Suitably, a highly expressed gene according to the invention encodes a protein that is expressed at high levels, and preferably specifically (i.e., substantially only, e.g., at a level at least about 100-fold greater than in other cells or tissues), in the target cell/tissue.

4. Construction of Synthetic Polynucleotides

The step of replacing synonymous codons for existing codons can be effected by any suitable technique. For example, in vitro mutagenesis methods can be employed which are known to those of skill in the art and include those described in Section 3.1 above.

The parent polynucleotide is preferably a natural gene. However, it is possible that the parent polynucleotide encodes a protein that is not naturally-occurring but has been engineered using recombinant techniques.

The parent polynucleotide need not be obtained from the plant but can be obtained from any suitable source, such as from a eukaryotic or prokaryotic organism. For example, the parent polynucleotide can be obtained from another plant or an animal. Broadly, the parent polynucleotide can be obtained from any eukaryotic or prokaryotic organism. In a preferred embodiment, the parent polynucleotide is obtained from a pathogenic organism. In such a case, a natural host of the pathogenic organism is preferably a plant. For example, the pathogenic organism can be a yeast, bacterium or virus

For example, suitable proteins which may be used for selective expression in accordance with the invention include, but are not limited to Bacillus thuringiensis (Bt) proteins as for example described by Hill et al. (1995, Euphytica 85(1-3): 119-123, incorporated herein by reference) and Nayak et al. (1997, Proc. Natl. Acad. Sci. USA 94(6): 2111-2116, incorporated herein by reference), sunflower seed albumin as for example described by Tabe et al. (1993, Genetica 90:181-200, incorporated herein by reference), herbicide resistance proteins such as the BAR protein as for example described by Thompson et al. (1987, EMBO J. 6:2519-2524, incorporated herein by reference).

The invention also contemplates synthetic polynucleotides encoding one or more desired portions of the protein to be expressed. A polynucleotide encodes a domain of the protein having a function associated therewith, or which is otherwise detectable, and preferably encodes at least 10, 20, 50, 100, 150, or 500 contiguous amino acid residues of the protein.

Regulatory polynucleotides which can be utilized to regulate expression from the synthetic polynucleotide include, but are not limited to, a promoter, an enhancer, and a transcriptional terminator. Such regulatory polynucleotides are known to those of skill in the art.

Suitable promoters which may be utilized to induce expression of the polynucleotides of the invention include constitutive promoters and inducible promoters. A particularly preferred promoter for dicotyledons which may be used to induce such expression is the Cauliflower Mosaic Virus (CaMV) 35S promoter. However, it will be appreciated that for monocotyledons, promoters including the ubiquitin promoter (P_{Ubi}) and the Emu promoter (p_{Emu}) may be employed.

Any suitable transcriptional terminator may be used which effects termination of transcription of polynucleotide. Preferably, the nopaline synthase (NOS) terminator, as for example disclosed in U.S. Pat. No. 5,034,322, is used as the transcription terminator.

4.1. Expression Vectors

Synthetic polynucleotides according to the invention can be operably linked to one or more regulatory polynucleotides in the form of an expression vector. The expression vector preferably contains an element(s) that permits stable integration of the vector into the host cell genome or autonomous replication of the vector in the cell independent of the genome of the cell. The vector may be integrated into the host cell genome when introduced into a host cell. For integration, the vector may rely on the foreign or endogenous DNA sequence or any other element of the vector for stable integration of the vector into the genome by homologous recombination. Alternatively, the vector may contain additional nucleic acid sequences for directing integration by homologous recombination into the genome of the host cell. The additional nucleic acid sequences enable the vector to be integrated into the host cell genome at a precise location in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAM.beta.1 permitting replication in Bacillus. The origin of replication may be one having a mutation to make its function temperature-sensitive in a Bacillus cell (see, e.g., Ehrlich, 1978, Proc. Natl. Acad. Sci. USA 75:1433).

4.2. Marker Genes

To facilitate identification of transformants, the synthatic construct desirably comprises a selectable or screenable marker gene as, or in addition to, the synthetic polynucleotide. The actual choice of a marker is not crucial as long as it is functional (i.e., selective) in combination with the plant cells of choice. The marker gene and the foreign or endogenous DNA sequence of interest do not have to be linked, since co-transformation of unlinked genes as, for example, described in U.S. Pat. No. 4,399,216 is also an efficient process in plant transformation.

Included within the terms selectable or screenable marker genes are genes that encode a “secretable marker” whose secretion can be detected as a means of identifying or selecting for transformed cells. Examples include markers that encode a secretable antigen that can be identified by antibody interaction, or secretable enzymes that can be detected by their catalytic activity. Secretable proteins include, but are not restricted to, proteins that are inserted or trapped in the cell wall (e.g., proteins that include a leader sequence such as that found in the expression unit of extensin or tobacco PR-S); small, diffusible proteins detectable, e.g. by ELISA; and small active enzymes detectable in extracellular solution (e.g., α-amylase, β-lactamase, phosphinothricin acetyltransferase).

4.2.1. Selectable Markers

A selectable marker is a gene the product of which provides for biocide resistance, resistance to heavy metals, prototrophy to auxotrophs, and the like. Examples of bacterial selectable markers are the dal genes from Bacillus subtilis or Bacillus licheniformis, or markers that confer antibiotic resistance such as ampicillin, kanamycin, erythromycin, chloramphenicol or tetracycline resistance. Exemplary selectable markers for selection of plant transformants include, but are not limited to, a hyg gene which encodes hygromycin B resistance; a neomycin phosphotransferase (neo) gene conferring resistance to kanamycin, paromomycin, G418 and the like as, for example, described by Potrykus et al. (1985, Mol. Gen. Genet., 199:183); a glutathione-S-transferase gene from rat liver conferring resistance to glutathione derived herbicides as, for example, described in EP-A 256 223; a glutamine synthetase gene conferring upon overexpression resistance to glutamine synthetase inhibitors such as phosphinothricin as, for example, described WO87/05327, an acetyl transferase gene from Streptomyces viridochromogenes conferring resistance to the selective agent phosphinothricin as, for example, described in EP-A 275 957, a gene encoding a 5-enolshikimate-3-phosphate synthase (EPSPS) conferring tolerance to N-phosphonomethylglycine as, for example, described by Hinchee et al. (1988, Biotech., 6:915), a bar gene conferring resistance against bialophos as, for example, described in WO91/02071; a nitrilase gene such as bxn from Klebsiella ozaenae which confers resistance to bromoxynil (Stalker et al., 1988, Science, 242:419); a methotrexate-resistant DHFR gene (Thillet et al., 1988, J. Biol. Chem., 263:12500); a mutant acetolactate synthase gene (ALS), which confers resistance to imidazolinone, sulfonylurea or other ALS-inhibiting chemicals (European Patent Application 154,204, 1985); a mutated anthranilate synthase gene that confers resistance to 5-methyl tryptophan; or a dalapon dehalogenase gene that confers resistance to the herbicide.

4.2.2. Screenable Markers

A screenable marker is a gene the product of which provides for a detectable trait. Preferred screenable markers include, but are not limited to, a uidA gene encoding a β-glucuronidase (GUS) enzyme for which various chromogenic substrates are known; a β-galactosidase gene encoding an enzyme for which chromogenic substrates are known; an aequorin gene (Prasher et al., 1985, Biochem. Biophys. Res. Comm., 126:1259), which may be employed in calcium-sensitive bioluminescence detection; a green fluorescent protein gene (Niedz et al., 1995 Plant Cell Reports, 14:403); a β-luciferase (luc) gene (Ow et al., 1986, Science, 234:856), which allows for bioluminescence detection; a β-lactamase gene (Sutcliffe, 1978, Proc. Natl. Acad. Sci. USA 75:3737), which encodes an enzyme for which various chromogenic substrates are known (e.g., PADAC, a chromogenic cephalosporin); an R-locus gene, encoding a product that regulates the production of anthocyanin pigments (red color) in plant tissues (Dellaporta et al., 1988, in Chromosome Structure and Function, pp. 263-282); an α-amylase gene (Ikuta et al., 1990, Biotech., 8:241); a tyrosinase gene (Katz et al., 1983, J. Gen. Microbiol., 129:2703) which encodes an enzyme capable of oxidizing tyrosine to DOPA and dopaquinone which in turn condenses to form the easily detectable compound melanin; or a xylE gene (Zukowsky et al., 1983, Proc. Natl. Acad. Sci. USA 80:1101), which encodes a catechol dioxygenase that can convert chromogenic catechols.

5. Introduction of the Synthetic Polynucleotide in a Plant Cell

The step of introducing the synthetic polynucleotide into a target cell or tissue will differ depending on the intended use and or species, and may involve infection by Agrobacterium tumefaciens or A rhizogenes, electroporation, micro-projectile bombardment or protoplast fusion.

A number of techniques are available for the introduction of DNA into a plant host cell. There are many plant transformation techniques well known to workers in the art, and new techniques are continually becoming known. The particular choice of a transformation technology will be determined by its efficiency to transform certain plant species as well as the experience and preference of the person practising the invention with a particular methodology of choice. It will be apparent to the skilled person that the particular choice of a transformation system to introduce a the synthetic polynucleotides of the invention into plant cells is not essential to or a limitation of the invention, provided it achieves an acceptable level of nucleic acid transfer. Guidance in the practical implementation of transformation systems for plant improvement is provided, for example, by Birch (1997, Annu. Rev. Plant Physiol. Plant Molec. Biol. 48: 297-326), which is incorporated herein by reference.

In principle both dicotyledonous and monocotyledonous plants that are amenable to transformation, can be modified by introducing a synthetic polynucleotide according to the invention into a recipient cell and growing a new plant that harbors and expresses the synthetic polynucleotide.

Introduction and expression of synthetic polynucleotides in dicotyledonous (broadleaved) plants such as tobacco, potato and alfalfa has been shown to be possible using the T-DNA of the tumor-inducing (Ti) plasmid of Agrobacterium tumefaciens (See, for example, Umbeck, U.S. Pat. No. 5,004,863, and International application PCT/US93/02480). A construct of the invention may be introduced into a plant cell utilizing A tumefaciens containing the Ti plasmid. In using an A. tumefaciens culture as a transformation vehicle, it is most advantageous to use a non-oncogenic strain of the Agrobacterium as the vector carrier so that normal non-oncogenic differentiation of the transformed tissues is possible. It is preferred that the Agrobacterium harbors a binary Ti plasmid system. Such a binary system comprises (1) a first Ti plasmid having a virulence region essential for the introduction of transfer DNA (T-DNA) into plants, and (2) a chimeric plasmid. The chimeric plasmid contains at least one border region of the T-DNA region of a wild-type Ti plasmid flanking the nucleic acid to be transferred. Binary Ti plasmid systems have been shown effective to transform plant cells as, for example, described by De Framond (1983, Biotechnology, 1:262) and Hoekema et al. (1983, Nature, 303:179). Such a binary system is preferred because it does not require integration into Ti plasmid in Agrobacterium.

Methods involving the use of Agrobacterium include, but are not limited to: (a) co-cultivation of Agrobacterium with cultured isolated protoplasts; (b) transformation of plant cells or tissues with Agrobacterium; or (c) transformation of seeds, apices or meristems with Agrobacterium.

Recently, rice and corn, which are monocots, have been shown to be susceptible to transformation by Agrobacterium as well. However, many other important monocot crop plants, including oats, sorghum, millet, and rye, have not yet been successfully transformed using Agrobacterium-mediated transformation. The Ti plasmid, however, may be manipulated in the future to act as a vector for these other monocot plants. Additionally, using the Ti plasmid as a model system, it may be possible to artificially construct transformation vectors for these plants. Ti plasmids might also be introduced into monocot plants by artificial methods such as microinjection, or fusion between monocot protoplasts and bacterial spheroplasts containing the T-region, which can then be integrated into the plant nuclear DNA.

In addition, gene transfer can be accomplished by in situ transformation by Agrobacterium, as described by Bechtold et al. (1993, C.R. Acad. Sci. Paris, 316:1194). This approach is based on the vacuum infiltration of a suspension of Agrobacterium cells.

Alternatively, foreign or chimeric nucleic acids may be introduced using root-inducing (Ri) plasmids of Agrobacterium as vectors.

Cauliflower mosaic virus (CaMV) may also be used as a vector for introducing of exogenous nucleic acids into plant cells (U.S. Pat. No. 4,407,956). CaMV viral DNA genome is inserted into a parent bacterial plasmid creating a recombinant DNA molecule that can be propagated in bacteria. After cloning, the recombinant plasmid again may be cloned and further modified by introduction of the desired nucleic acid sequence. The modified viral portion of the recombinant plasmid is then excised from the parent bacterial plasmid, and used to inoculate the plant cells or plants.

Synthetic polynucleotides according to the invention can also be introduced into plant cells by electroporation as, for example, described by Fromm et al. (1985, Proc. Natl. Acad. Sci., U.S.A, 82:5824) and Shimamoto et al. (1989, Nature 338:274-276). In this technique, plant protoplasts are electroporated in the presence of vectors or nucleic acids containing the relevant nucleic acid sequences. Electrical impulses of high field strength reversibly permeabilize membranes allowing the introduction of nucleic acids. Electroporated plant protoplasts reform the cell wall, divide and form a plant callus.

Another method for introducing synthetic polynucleotides into a plant cell is high velocity ballistic penetration by small particles (also known as particle bombardment or microprojectile bombardment) with the nucleic acid to be introduced contained either within the matrix of small beads or particles, or on the surface thereof as, for example described by Klein et al. (1987, Nature 327:70). Although, typically only a single introduction of a new nucleic acid sequence is required, this method particularly provides for multiple introductions.

Alternatively, the synthetic polynucleotides can be introduced into a plant cell by contacting the plant cell using mechanical or chemical means. For example, a nucleic acid can be mechanically transferred by microinjection directly into plant cells by use of micropipettes. Alternatively, a nucleic acid may be transferred into the plant cell by using polyethylene glycol which forms a precipitation complex with genetic material that is taken up by the cell.

There are a variety of methods known currently for transformation of monocotyledonous plants. Presently, preferred methods for transformation of monocots are microprojectile bombardment of explants or suspension cells, and direct DNA uptake or electroporation as, for example, described by Shimamoto et al. (1989, supra). Transgenic maize plants have been obtained by introducing the Streptomyces hygroscopicus bar gene into embryogenic cells of a maize suspension culture by microprojectile bombardment (Gordon-Kamm, 1990, Plant Cell, 2:603-618). The introduction of genetic material into aleurone protoplasts of other monocotyledonous crops such as wheat and barley has been reported (Lee, 1989, Plant Mol. Biol. 13:21-30). Wheat plants have been regenerated from embryogenic suspension culture by selecting only the aged compact and nodular embryogenic callus tissues for the establishment of the embryogenic suspension cultures (Vasil, 1990, Bio/Technol. 8:429-434). The combination with transformation systems for these crops enables the application of the present invention to monocots. These methods may also be applied for the transformation and regeneration of dicots. Transgenic sugarcane plants have been regenerated from embryogenic callus as, for example, described by Bower et al. (1996, Molecular Breeding 2:239-249).

Alternatively, a combination of different techniques may be employed to enhance the efficiency of the transformation process, e.g., bombardment with Agrobacterium coated microparticles (EP-A-486234) or microprojectile bombardment to induce wounding followed by co-cultivation with Agrobacterium (EP-A-486233).

The literature articles referred to above are all incorporated herein by reference.

6. Production and Characterisation of Differentiated Transgenic Plants

6.1. Regeneration

The methods used to regenerate transformed cells into differentiated plants are not critical to this invention, and any method suitable for a target plant can be employed. Normally, a plant cell is regenerated to obtain a whole plant from a transformation process.

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxin and cytokinins. It is sometimes advantageous to add glutamic acid and proline to the medium, especially for such species as corn and alfalfa. Efficient regeneration will depend on the medium, on the genotype, and on the history of the culture. If these variables are controlled, regeneration is reproducible. Regeneration also occurs from plant callus, explants, organs or parts. Transformation can be performed in the context of organ or plant part regeneration as, for example, described in Methods in Enzymology, Vol. 118 and Klee et al. (1987, Annual Review of Plant Physiology, 38:467), which are incorporated herein by reference. Utilizing the leaf disk-transformation-regeneration method of Horsch et al. (1985, Science, 227:1229, incorporated herein by reference), disks are cultured on selective media, followed by shoot formation in about 2-4 weeks. Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature transgenic plants are propagated by the taking of cuttings or by tissue culture techniques to produce multiple identical plants. Selection of desirable transgenotes is made and new varieties are obtained and propagated vegetatively for commercial use.

In seed propagated crops, the mature transgenic plants can be self-crossed to produce a homozygous inbred plant. The inbred plant produces seed containing the newly introduced foreign gene(s). These seeds can be grown to produce plants that would produce the selected phenotype, e.g., early flowering.

Parts obtained from the regenerated plant, such as flowers, seeds, leaves, branches, fruit, and the like are included in the invention, provided that these parts comprise cells that have been transformed as described. Progeny and variants, and mutants of the regenerated plants are also included within the scope of the invention, provided that these parts comprise the introduced synthetic polynucleotides of the invention.

It will be appreciated that the literature describes numerous techniques for regenerating specific plant types and more are continually becoming known. Those of ordinary skill in the art can refer to the literature for details and select suitable techniques without undue experimentation.

6.2. Characterisation

To confirm the presence of the synthetic polynucleotide in the regenerating plants, a variety of assays may be performed. Such assays include, for example, “molecular biological” assays well known to those of skill in the art, such as Southern and Northern blotting and PCR; a protein expressed by the synthetic polynucleotide may be analysed by high performance liquid chromatography or ELISA (e.g., nptII) as is well known in the art.

7. Applications

In one embodiment of the present invention, the target cell is suitably a cell or tissue of a plant leaf. Advantageously, the protein which is desired to be selectively expressed in the leaf cell or tissue is not expressible in a cell or tissue of the plant's root from a parent polynucleotide at a level sufficient to effect a particular function associated with said protein. In this embodiment, the step of replacing at least one existing codon with a synonymous codon is characterized in that the synonymous codon has a higher translational efficiency in the leaf cell or tissue compared to the root cell or tissue. Accordingly, a synthetic polynucleotide is produced having altered translational kinetics compared to the parent polynucleotide wherein the protein is expressible in the leaf cell or tissue at a level sufficient to effect a particular function associated with said protein, but wherein the protein is not expressible in the root cell or tissue at a level sufficient to effect said function.

The above embodiment may be utilized advantageously for conferring herbicide resistance in leaves of a plant, but not in roots of the plant where overexpression of a herbicide resistance gene may adversely affect plant growth. In such a case, a suitable protein may include the BAR protein which confers resistance to the herbicide BASTA.

8. Expressing a Protein in a Target Cell or Tissue by In Vivo Expression of Iso tRNAs in the Target Cell or Tissue

The invention also extends to a method wherein a protein can be selectively expressed in a target cell of a plant by introducing into the cell an auxiliary polynucleotide capable of expressing in the target cell one or more isoaccepting transfer RNAs which are not normally expressed in relatively high abundance in the target cell but which are rate-limiting for expression of the protein from a parent polynucleotide to a level sufficient for effecting a function associated with the protein. In this embodiment, introduction of the auxiliary polynucleotide sequence in the target cell changes the translational kinetics of the parent polynucleotide such that said protein is expressed at a level sufficient to effect a function associated with the protein.

The step of introducing the auxiliary polynucleotide sequence into the target cell or a tissue comprising a plurality of these cells can be effected by any suitable means. For example, analogous methodologies for introduction of the synthetic polynucleotide referred to above can be employed for delivery of the auxiliary polynucleotide into said target cell.

In practice, the choice of iso-tRNA supplied to a target cell using this method depends on whether the target cell in which protein expression is desired has a low or high abundance of that iso-tRNA and on whether the parent polynucleotide comprises codons corresponding to that iso-tRNA species. Thus, an iso-tRNA is supplied to the target cell by the auxiliary polynucleotide when that iso-tRNA is in relatively low abundance in the target cell and when parent polynucleotide comprises codons corresponding to that iso-tRNA species.

The invention is further described with reference to the following non-limiting examples.

### EXAMPLE 1

Construction of expression vectors for determining relative abundance of different iso-tRNAs in different cells or tissues of Arabidopsis thaliana.

An oligonucleotide (SEQ ID NO: 23; sequence complementary to the termination codon for GFP, is underlined), and a suite of oligonucleotides (SEQ ID NO: 24, 25, 26, 27, 28, 29, 30, 31, 32, 33 and 34; the first codon of GFP, is underlined) will be synthesized and used for PCR amplification of pBIN m-gfp5-ER⁻RS⁻ (SEQ ID NO: 35) template with Taq DNA polymerase (Amplification parameters: 94° C./1 min; 55° C./1 min; 72° C./2 min for 30 cycles). The amplified fragments will have nucleic acid sequences as shown in SEQ ID NO: 1, 3, 5, 7, 9, 11, 13, 15, 17, 19, and 21. The deduced amino acid sequences encoded by these synthetic fragments are shown below the corresponding nucleic acid sequences as well as in SEQ ID NO: 2, 4, 6, 8, 10, 12, 14, 16, 18, 20 and 22. In summary, the synthetic fragments contain an artificial start codon followed by a tandem repeat of six identical codons specific for a given iso-tRNA species. The tandem repeat immediately precedes the second codon of the gfp gene. The synthetic fragments by SEQ ID NO and tandem repeat encoded thereby are presented in the TABLE 2.

TABLE 2
Synthetic fragments and tandem repeats encoded thereby.

| SEQ ID NO | Tandem repeat |
|---|---|
| 1 | control |
| 3 | His (CAT) × 6 |
| 5 | His (CAC) × 6 |
| 7 | Leu (CTA) × 6 |
| 9 | Leu (CTC) × 6 |
| 11 | Leu (CTG) × 6 |
| 13 | Leu (CTT) × 6 |
| 15 | Leu (TTA) × 6 |
| 17 | Leu (TTG) × 6 |
| 19 | Lys (AAA) × 6 |
| 21 | Lys (AAG) × 6 |

The amplified fragments will be cloned using either the HindIII or BamHI sites at the 5′ end and either the PstI or EcoRI sites at the 3′ end of the pAOV2 polylinker (Mylne and Botella, 1999, Plant Mol. Biol. Res., in press; the manuscript relating thereto is annexed hereto as Annexure A) for expression in Escherichia coli and for plant transformation experiments.

### EXAMPLE 2

Agrobacterium Tri-Parental Mating

Tri-parental mating uses natural conjugation between bacteria to transfer a plasmid from E. coli (donor) to Agrobacterium (recipient) with the conjugal assistance from (helper) E. coli (HB101/pRK2013). Successful conjugation is selected for by combinatorial selection with antibiotics.

Agrobacterium Media:

There are a number of possible media that can be used for Agrobacterium growth including (Minimal A, LB, YEP, YEB, YM etc). Some of these media provide additional selection for Agrobacterium by using sucrose as the primary carbon source rather than glucose. E. coli cannot metabolize sucrose well. The following procedures all involve use of LB for Agrobacterium growth. A superscript numeral following an antibiotic name or abbreviated antibiotic name refers to the amount of antibiotic used in the growth media in micrograms per milliliter.

Procedure:

Plates are made for recipient Agrobacterium (LBA4404: LB-Rif⁵⁰/Strep²⁵ or GV3101: LB-Rif⁵⁰/Gen²⁵-Rif⁵⁰ alone); for donor (e.g. LB-Tet¹⁰ for pAOV2—plates depend on the vector used) and for helper (LB-Kan⁵⁰); as well an LB (only) plate for the mating. Plates are also made that contain antibiotics for the vector to be used as well as for Agrobacterium (e.g. LB-Rif⁵⁰/Tet²).

The bacteria are grown on the plates (start Agrobacterium first) until good sized individual colonies are formed. A loop is used to take a colony from each plate and these colonies are mixed together in the middle of the LB plate. This plate is then incubated at 28° C. for 24 hours.

A loop of the mix is then plated out (16 streak) onto a combinatorial selection plate. This plate is then placed in a 28° C. incubator for 2-3 days after which isolated individual colonies are picked for culture (at this stage, some of the bacteria may be used to screen for recombinant Agrobacterium by PCR)—Note it is important to “ease” the bacteria back onto selection (LB-Rif⁵⁰/Tet²/Strep²⁵ or Gen²⁵ all at once after mating is too strong and no colonies will grow back from the mating).

A colony from the above plate is then exposed to stronger selection before subsequent growth and PCR (e.g. plates containing LB-Rif¹⁰⁰/Tet²/Strep²⁵)

Control Plate:

If tri-parental mating is desired to be validated, a control plate may be run in tandem—i.e. LB plate with only two of D, H or R (D—donor; H—helper; R—recipient). This plate is incubated at 28° C. for 24 hours. A loop of the mixes is then plated out (16 streak) onto a combinatorial selection plate. And the plate is then placed at 28° C. for 2-3 days—no colonies should be obtained (however, a few colonies may be obtained for the DR—because of a low rate of conjugation). This control validates that your helper indeed helps and that the combinatorial selection is working.

### EXAMPLE 3

Screening Agrobacterium by PCR

A portion of a large (matchhead-size) Agrobacterium colony is used to inoculate selective media. 20 μl of the following PCR PreMix is then placed into a PCR tube. The commercial primers used in the PCR PreMix are custom oligonucleotides made to specification by Operon Technologies, Inc. The sequence of the 35S forward primer is 5′-CCTTCGCAAGACCCTTCCTC-3′ (SEQ ID NO: 37). The sequence of the NOS reverse primer is 5′-TAGTTTGCGCGCTATATTTTG-3′ (SEQ ID NO: 38).

PCR PreMix

| | |
|---|---|
| dNTP's @ 10 mM | 50 μL |
| Commercial 35S Forward Primer @ 10 μM | 50 μL |
| Commercial NOS Reverse Primer @ 10 μM | 50 μL |
| MgCl₂ @ 25 mM | 150 μL |
| 10X PCR Buffer II | 250 μL |
| H₂O | 1450 μl |

A Gilson Pipetteman™ P₁₀ tip is used to stab the same Agrobacterium colony from the plate and the stab of bacteria is mixed with 20 μL of the PreMix. Two microliters (2 Units) of Taq DNA polymerase, 3 μL of H₂O and 40 μL of mineral oil are then added to the mixture.

Mixtures are then amplified by PCR using the following parameters:

| | | |
|---|---|---|
| 94° C. | 1 min | |
| 94° C. | 30 sec | 35 cycles |
| 56° C. | 30 sec | |
| 72° C. | 3 min | |
| 72° C. | 15 min | |
| 4° C. | HOLD | |

After completion, 10 μL of the PCR reaction is then subjected to agarose gel electrophoresis to confirm whether PCR products have been obtained.

### EXAMPLE 4

Transformation of Arabidopsis by Vacuum Infiltration (in Planta)

Vacuum infiltration involves putting adult Arabidopsis plants under a suspension of Agrobacterium in a vacuum chamber. A vacuum is drawn and the air spaces in the plant expand and bubble to the surface. When the pressure is returned, the Agrobacterium solution replaces the air spaces and the Agrobacterium are introduced into the inside of the plant. The Agrobacterium transform, among other cells, parts of the floral meristem and some of these cells will give rise to embryos and eventually, when planted, a whole transgenic plant.

Arabidopsis Preparation

An 8 cm diameter pot is filled with a moist soil mixture. The pot is covered with a 15 cm diameter (flyscreen) mesh circle, and an elastic band is used to secure it, keeping the mesh taught across the top, and making sure that the soil touches the mesh.

Mancozeb 2 g/L (Yates Mancozeb Plus Garden Fungicide) is sprayed onto the soil and Arabidopsis (ecotype Columbia)—approximately 20 or so per pot—is sprinkled thereon.

The pots are placed into a Yates seedling chamber (which holds about 16 pots), and a lid is placed upon the containers. The containers are then placed in a 4° C. cold room for 3-5 days for stratification (the cold snap synchronizes germination).

The plants are then placed under short daylights (i.e., 8 hr day) for 10-14 days (short day encourages vegetative growth). Seedlings may be thinned out if required. However, the plants should be kept well watered when young.

Pots are then placed in long day (i.e. 16 hr day) to induce bolting. The plants are then watered from below about every 2-3 days (plants require more water when they are filling their siliques). Bolts may be pruned back if necessary to encourage numerous unopened buds at time of infiltration (by clipping primary inflorescence at its base, and letting secondary inflorescences grow until they start to show open flowers).

The above method has been used successfully to transform mature plants and even non-bolted plants. However, it will be appreciated that plants should not be water stressed, otherwise their stomata will be shut and infiltration will not be efficient. Plants should be watered the evening before to ensure the stomata are open for the infiltration. It will be appreciated that healthy plants and correct infiltration media are important to a successful transformation.

Agrobacterium Preparation

When the plants begin to bolt, Agrobacterium-pAOV2 constructs are plated onto LB-Rif⁵⁰Tet² at 28° C. A 5 mL LB-Rif⁵⁰Tet² culture is set up with fresh Agrobacterium from the plates and shaken at 28° C. for 2 days. The culture is then added to a 100 mL Erlenmeyer flask containing 35 mL LB-Rif⁵⁰Tet² and this is shaken overnight before addition to a 1 L Erlenmeyer flask containing 400 mL LB-Tet² (No Rif) and subsequent culturing at 28° C. for about 24 hrs (or until Agrobacterium is in late log/early stationary phase)

Infiltration

The Agrobacterium culture (ideal OD₆₀₀ is about 1.6-2.0) is then harvested by centrifugation in a GSA rotor (Sorvall) at 5000 rpm for 20 min at room temperature (however, infiltration still works if centrifugation is performed at 4° C.).

The resulting cell pellet is resuspended in 1 L infiltration media (see below). Some of the infiltration culture is poured into two 1 L containers which are then placed in the vacuum chamber. A skewer is placed through opposite holes in the bottom of two pots containing A. thaliana and the pots are inverted in the containers. Each pot should be suspended upside-down in a bottle with the skewer holding the pot a few centimeters from the bottom. In this regard, it is important to ensure that the entire bolts of the plants are covered by the culture (especially the flowers)—fungal infection may result if too much infiltration media enters the soil and rosette.

The vacuum chamber is then closed, before closing the air outlet of the chamber, and drawing a vacuum until solution bubbles vigorously. The vacuum is then released as soon as possible.

The infiltration procedure is then repeated on the same plants.

Plants are placed on their sides in the containers. The top of the containers are then covered with a plastic wrap before placing the containers under long day light for one night. The next day the plastic wrap is removed and the plants are set upright.

Plants should not be watered until approximately 6 days after infiltration (although plants should be watered if they are looking wilted).

Plants are then grown until bolts start to yellow on some siliques. Seed catchers are then placed on the plants. For example, seed catchers may be made from soft-drink bottles by cutting off the bottom of the bottles and making a hole in each bottle near the neck (about where the curve begins to straighten out into the cylinder of the bottle).

When flowers terminate into siliques, watering of the plants is stopped and when the entire plant is dry, the seeds are harvested (seeds from green siliques contain germination inhibitors)

Basta® Selection of Transformants

Approximately 0.5 mL of seed is planted in a seedling tray containing moist soil, and this is covered with vermiculite dust and a chamber lid before placing at 4° C. to synchronize germination (3-5 days).

The chambers are placed under long day light and at emergence, the plants are sprayed with 2000 mg/L (active component of Basta®—is glufosinate ammonium at a concentration of 200 g/L in Hoechst Horticulture Basta® U.N. No 2902).

The plants are then sprayed three days later and again three days after that. Transgenic plants will be very green and much larger than non-transgenic plants which should wither and die. Transgenic plants can be transferred later to individual pots if desired.

Routinely, approximately 75-100 transformants from five plants are isolated using this procedure.

Solution and media used for infiltration are presented in TABLE 3.

TABLE 3
Solutions and Media

| Infiltration media | 1 liter | 2 liters | 5 liters |
|---|---|---|---|
| 0.5x MS salts | 2.16 g | 4.32 g | 10.8 g |
| 5% w/v Sucrose | 50 g | 100 g | 250 g |
| 1x B5 (or MS) vitamins | 1 mL @ | 2 mL @ | 5 mL @ |
| | 1000X | 1000X | 1000X |
| 0.25 g/L MES | 0.25 g | 0.5 g | 1.25 g |
| pH to 5.7 (with KOH) | | | |
| After autoclaving add: | | | |
| 10 μL/L of 1 mg/mL BAP | | | |
| 50 μL/L Silwet L-77 | | | |
| (detergent) | | | |

### EXAMPLE 5

Confocal Microscopy

Transgenic Arabidopsis seedlings are grown in sterile agar culture for 5 days, and are mounted in water under glass coverslips for microscopy. The specimens are examined using a Bio-Rad MRC-600 laser-scanning confocal microscope equipped with a krypton-argon laser and filter sets suitable for the detection of fluorescein and Texas red dyes (Bio-Rad KlyK2), and a Nikon 603 PlanApo numerical aperture 1.2 water-immersion objective. Dual-channel confocal images and video montages of seedlings are suitably composed using ADOBE PhotoShop.

### EXAMPLE 6

This example is from J. Mylne et al, 1998, Genetics Resources 16:257-262, and is based on our interest in devising methods to identify functions for the vast numbers of Arabidopsis genes now available. For this purpose we have constructed a set of binary vectors that will allow the quick production of transgenic Arabidopsis plants containing either sense or antisense copies of EST clones obtained from the PRL2 library (Newman et al., 1994 Plant Physiol. 106:1241-1255). These vectors are based on the pSLJ series (Jones et al., 1992, Transgen. Res. 1:285-297) containing the bialophos resistance (BAR) gene that confers resistance to the herbicide BASTA (Thompson et al, 1987, EMBO J. 6:2519-2524). In addition, our vectors contain a 35S CaMV promoter-polylinker-nos terminator cassette that allows the direct cloning of Arabidopsis ESTs in either antisense (pAOV and pAOV2) or sense (pSOV and pSOV2) orientation. We also describe the construction of two additional vectors conferring BASTA resistance and containing the pBluescript polylinker in both orientations inserted between the 35S CaMV promoter and nos terminator (pKMB and pSMB). The following abbreviations are used in this Example: ABRC, Arabidopsis biological resource center; CaMV, cauliflower mosaic virus; EST, expressed sequence tag; NASC, Nottingham Arabidopsis stock centre; PCR, polymerase chain reaction.

Thanks to the ongoing plant genome projects, the emphasis in molecular biology has shifted from cloning genes to establishing the function of available genes. Among the many methods available to study gene function, two of the most popular ones are the production of loss-of-function and/or gain-of-function mutants. When a laboratory clones a new gene of unknown function, one of the first approaches is to knock out the gene and study the phenotype of the resulting mutant plants. Down-regulation or over-expression of a given gene is most frequently achieved by the production of transgenic plants carrying antisense or extra sense copies of the gene. Unfortunately, not all plants are amenable to transformation and not all genes can be studied by this method.

The main considerations when planning to produce transgenic plants carrying either sense or antisense constructs are (a) the species to uses (b) the transformation method and (c) the selectable marker. Arabidopsis is the model system used by many laboratories due to the simplicity of its genome and availability of standard methods for transformation. Recent developments in arabidopsis transformation by in planta vacuum infiltration (Bechtold et al., 1993, Comptes Rendus de l'Academie des Sciences Serie III Sci 316:1194-1199) have avoided the need for tissue culture, providing an extremely easy method to obtain transgenic plants without the help of specialized equipment. Furthermore, the availability of herbicide resistance genes (De Block et al., 1987, EMBO J 6:2513-2518; Lee et al., 1988, EMBO J 7:1241-1248) has superseded the need to use antibiotic resistance as selectable marker and sterile techniques for the selection of transformants.

Our vectors take advantage of all the above mentioned developments. They allow direct cloning of ESTs (Newman et al., 1994, Plant Physiol. 106:1241-1255) in either sense or antisense orientations that can be combined with in planta transformation and direct selection of transgenic plants in seed trays by BASTA resistance (Thompson et al., 1987, EMBO J 6:2519-2524).

Materials and Methods

Standard techniques were used for bacterial growth (E. coli DH10B) and DNA manipulation (Sambrook et al., 1989, Molecular Cloning: a Laboratory Manual, 2nd edition, Cold Spring Harbor Laboratory Press, NY). Large amounts of high purity binary vector DNA required for cloning or sequencing were obtained as de-scribed by Jones et al. (1992, Transgen. Res. 1:285-297).

Vector Construction

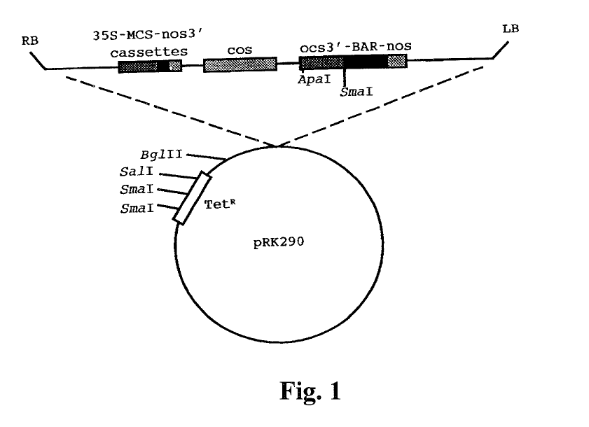

All vectors described in this paper have been constructed using pSLJ75515 as backbone and their general outlay is shown in FIG. 1.

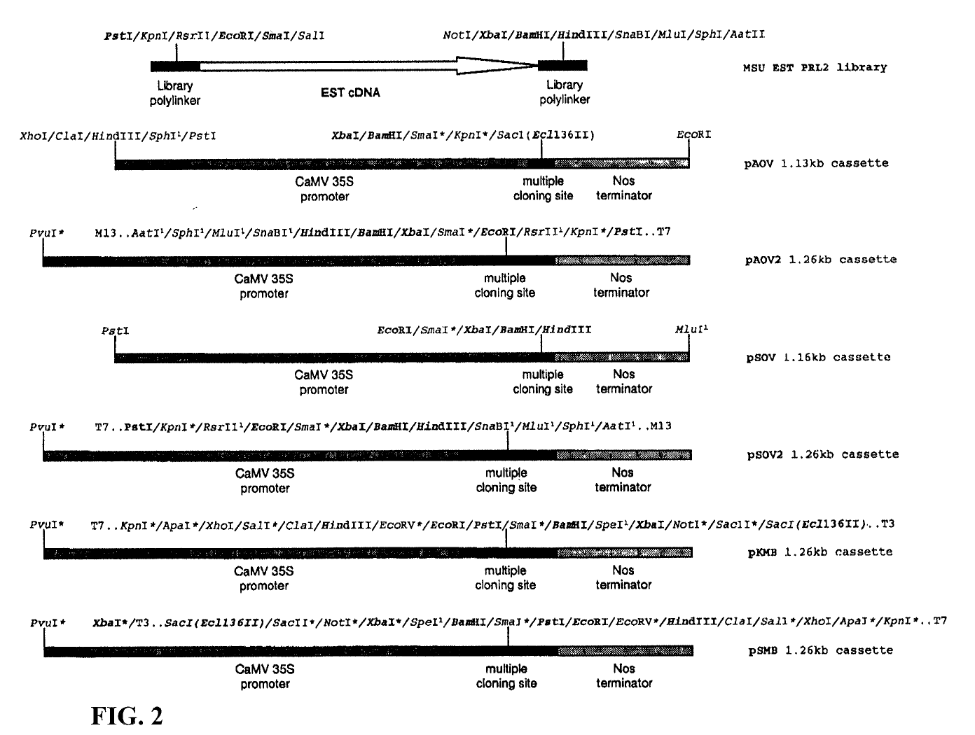

pAOV2 (Antisense Orientation Vector) (FIG. 2) was constructed by separately excising the 35S CaMV promoter and nos terminator from pBII21 (Bevan, 1984, Nucleic Acids Res. 12:8711-8721) and ligating them into the pUC18 multiple cloning site. This 35S-polylinker-nos cassette was cloned into pSLJ75515 which had previously had superfluous restriction enzyme sites removed.

pSOV (Sense Orientation Vector) (FIG. 2) was constructed by separately excising the 35S CaMV promoter and nos terminator from pBII21 and ligating them into pZLD. pZLD was obtained by removing the EST insert from a PRL2 clone with Sal I and Not I. The ends of the vector were then blunted and the vector religated; as a result pZLD is missing the restriction sites from Sal I to Not I. The 35S-polylinker-nos cassette was cloned into pSLJ75515 which had previously had superfluous restriction enzyme sites removed.

pAOV2 and pSOV2 (FIG. 2) were constructed using pAOV as backbone. The pZLD multiple cloning site was amplified by PCR using T7 and M13 primers and ligated into pAOV that had been previously modified to remove several conflicting restriction sites.

pKMB and pSMB (FIG. 2) were also constructed using pAOV as back-bone. The pBluescript multiple cloning site was amplified by PCR using T3 and T7 primers and ligated into pAOV that had been previously modified to remove several conflicting restriction sites. A detailed construction strategy flowchart is available and will be included with any vectors supplied.

Results and Discussion

pAOV allows the direct insertion of EST clones from the PRL2 library into a binary plasmid under the control of the 35S CaMV promoter and the nos terminator sequences. EST clones obtained from either the Arabidopsis Biological Research Center (ABRC, Ohio State) or the Nottingham Arabidopsis Stock Centre (NASC, Nottingham) as bacterial stab cultures can be grown, miniprepped and the inserts excised with Sma I at the 5′ end and either Xba I or BamHI at the 3′ end. DNA bands purified from gels can be ligated into pAOV using a sticky/blunt ligation strategy (Xba I or BamHI at 5′ and Ecl136II at 3′). We have successfully prepared 80 antisense constructs with pAOV of which 40 have already been used to transform arabidopsis by vacuum infiltration. We have just recently finished the construction of pAOV2 that contains several extra restriction sites in the polylinker allowing the use of either EcoR I or Pst I at the 5′ side of the EST clone and Xba I, BamHI or Hind III at the 3′ end. pAOV2 will allow a more efficient sticky end ligation and provides several different possibilities for cloning.

pSOV was designed to allow the cloning of EST sequences from the arabidopsis PRL2 library in sense orientation. The purpose of this vector is the production of plants with extra copies of a particular gene to achieve either over-expression of the encoded protein or the overall down regulation of the gene by co-supression events. EST sequences can be cloned using the EcoRI site at the 5′ end of the EST clone and the Xba I, BamH I or Hind III sites at the 3′ end. We have also finished the construction of pSOV2 that contains an extra Pst I restriction site at the 5′ end of the polylinker providing extra choices for the cloning strategy. There are no spurious ATG codons in either pSOV or pSOV2 that could interfere with the start of translation of the encoded protein.

Finally we have constructed pKMB and pSMB, two binary vectors also based on the pSLJ75515 backbone. Both vectors contain the 35S CaMV promoter and nos terminator sequences separated by the complete pBluescript polylinker in KS (pKMB) or SK (pSMB) orientations, including the T3 and T7 promoters. Please note that the Xba I site in pSMB occurs twice within the polylinker.

In conclusion, our collection of binary vectors will allow the quick production of sense and antisense constructs containing arabidopsis ESTs in no more than a week after obtaining the E. coli cultures from any of the repository sites. They provide alternative strategies for the cloning, allowing for the possibility of the EST clone containing some of the designated restriction sites within its sequence. Using the vectors described here we routinely complete the process of obtaining an EST clone, inserting it into the binary vector in sense and/or antisense orientations, perform in planta transformation and select BASTA resistant seedlings in six weeks. Nevertheless, the use of these vectors is of course not limited to ESTs, especially for the pKMB and pSMB vectors.

The disclosure of every patent, patent application, and publication cited herein is hereby incorporated by reference in its entirety.

The present invention has been described in terms of particular embodiments found or proposed by the present inventors to comprise preferred modes for the practice of the invention. Those of skill in the art will appreciate that, in light of the present disclosure, numerous modifications and changes can be made in the particular embodiments exemplified without departing from the scope of the invention. All such modifications are intended to be included within the scope of the appended claims.

What is claimed is:

1. A method of constructing a synthetic polynucleotide from which a protein is selectively expressible in a target cell of a plant, relative to another cell of the plant, said method comprising:
selecting a first codon of a parent polynucleotide for replacement with a synonymous codon which has a higher translational efficiency in said target cell than in said other cell; and
replacing said first codon with said synonymous codon to form said synthetic polynucleotide.

2. The method of claim 1, wherein said first codon and said synonymous codon are selected by:
comparing translational efficiencies of individual codons in said target cell relative to said other cell; and
selecting said first codon and said synonymous codon based on said comparison.

3. The method of claim 2, wherein the translational efficiency of an individual codon is measured by:
introducing into said target cell and into said other cell, a synthetic construct comprising a reporter polynucleotide fused in frame with a tandem repeat of said individual codon, wherein said reporter polynucleotide encodes a reporter protein, and wherein said synthetic construct is operably linked to a regulatory polynucleotide; and
comparing expression of said reporter protein in said target cell and in said other cell to determine the translational efficiency of said individual codon in said target cell relative to said other cell.

4. The method of claim 3, further comprising:
introducing the synthetic construct into a progenitor cell of a cell selected from the group consisting of said target cell and said other cell; and
producing said target cell from said progenitor cell, wherein said cell contains said synthetic construct.

5. The method of claim 3, further comprising:
introducing the synthetic construct into a progenitor cell of a cell selected from the group consisting of said target cell and said another cell; and
growing a plant or part thereof from said progenitor cell, wherein said plant or part thereof comprises said cell containing said synthetic construct.

6. The method of claim 3, further comprising: introducing the synthetic construct into a plant or part thereof such that said synthetic construct is introduced into said target cell or said other cell.

7. The method of claim 2, wherein said synonymous codon corresponds to a reporter construct from which the reporter protein is expressed in said target cell at a level that is at least 110% the level of the reporter protein that is expressed from the same reporter construct in said other cell.

8. The method of claim 2, wherein the translational efficiency of an individual codon is compared by measuring the abundance of an iso-tRNA corresponding to said individual codon in said target cell relative to said other cell.

9. The method of claim 8, wherein said synonymous codon corresponds to an iso-tRNA which is in higher abundance in said target cell relative to said other cell.

10. The method of claim 8, wherein selecting said first codon and said synonymous codon comprises:
measuring abundance of different iso-tRNAs in said target cell relative to said other cell; and
selecting said first codon and said synonymous codon based on said measurement, wherein said synonymous codon corresponds to an iso-tRNA which is in higher abundance in said target cell than in said other cell.

11. The method of claim 8, wherein said synonymous codon corresponds to an iso-tRNA that is present in said target cell at a level which is at least 110% of the level of the iso-tRNA that is present in said other cell.

12. The method of claim 1, wherein said synonymous codon is selected from the group consisting of (1) a codon used at relatively high frequency by genes of said target cell, (2) a codon used at relatively high frequency by genes of the plant, (3) a codon used at relatively low frequency by genes of said other cell, and (4) a codon used at relatively low frequency by genes of another organism.

13. The method of claim 1, wherein said first codon is selected from the group consisting of (a) a codon used at relatively high frequency by genes of said other cell, (b) a codon used at relatively low frequency by genes of said target cell, (c) a codon used at relatively low frequency by genes of the plant, and (d) a codon used at relatively high frequency by genes of another organism.

14. The method of claim 1 wherein said first codon and said synonymous codon are selected such that said protein is expressed from said synthetic polynucleotide in said target cell at a level which is at least 110% of the level at which said protein is expressed from said parent polynucleotide in said target cell.

15. The method of claim 1, wherein said other cell is a precursor cell of said target cell.

16. The method of claim 1, wherein said other cell is a cell derived from said target cell.

17. A synthetic polynucleotide constructed according to the method of claim 1.

18. A method of selectively expressing a protein in a target cell of a plant, said method comprising:
replacing a first codon of a parent polynucleotide encoding said protein with a synonymous codon to produce a synthetic polynucleotide having altered translational kinetics compared to said parent polynucleotide, such that said protein is expressed in said target cell, but such that said protein is not substantially expressed in another cell of the plant; and
introducing into a cell selected from the group consisting of said target cell and a precursor of said target cell, said synthetic polynucleotide operably linked to a regulatory polynucleotide,
whereby said protein is selectively expressed in said target cell.

19. The method of claim 18, wherein said synonymous codon has a higher translational efficiency in said target cell than in said other cell.

20. A method of expressing a protein in a target cell of a plant from a first polynucleotide, said method comprising:
introducing into a cell selected from the group consisting of said target cell and a precursor of said target cell, a second polynucleotide encoding an iso-tRNA, wherein said second polynucleotide is operably linked to a regulatory polynucleotide, and wherein said iso-tRNA is normally in relatively low abundance in said target cell and corresponds to a codon of said first polynucleotide, whereby said protein is expressed in said target cell.

21. A vector comprising the synthetic polynucleotide of claim 17.

22. A cell comprising the synthetic polynucleotide of claim 17.

23. A cell comprising the vector of claim 21.

24. A cell produced by the method of claim 20.

25. The method of claim 1, wherein said protein is not substantially expressed in said other cell.

26. The method of claim 2, wherein said tandem repeat comprises at least three copies of said individual codon.

27. A transgenic plant or plant part comprising a cell containing the synthetic polynucleotide of claim 17.


SEQUENCE LISTING (38 sequences)

SEQ ID NO: 1 - DNA, 762 bp, Artificial Sequence
  GFP control construct
tttaagcttggatcccaaggagatataacaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 2 - protein, 238 aa, Artificial Sequence
  GFP control construct
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 3 - DNA, 780 bp, Artificial Sequence
  His(CAT)6 GFP construct
tttaagcttggatcccaaggagatataacaatgcatcatcatcatcatcatagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 4 - protein, 244 aa, Artificial Sequence
  His(CAT)6 GFP construct
MHHHHHHSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 5 - DNA, 780 bp, Artificial Sequence
  His(CAC)6 GFP construct
tttaagcttggatcccaaggagatataacaatgcaccaccaccaccaccacagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 6 - protein, 244 aa, Artificial Sequence; identical to SEQ ID NO: 4
  His(CAC)6 GFP construct

SEQ ID NO: 7 - DNA, 780 bp, Artificial Sequence
  Leu(CTA)6 GFP construct
tttaagcttggatcccaaggagatataacaatgctactactactactactaagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 8 - protein, 244 aa, Artificial Sequence
  Leu(CTA)6 GFP construct
MLLLLLLSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 9 - DNA, 780 bp, Artificial Sequence
  Leu(CTC)6 GFP construct
tttaagcttggatcccaaggagatataacaatgctcctcctcctcctcctcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 10 - protein, 244 aa, Artificial Sequence; identical to SEQ ID NO: 8
  Leu(CTC)6 GFP construct

SEQ ID NO: 11 - DNA, 780 bp, Artificial Sequence
  Leu(CTG)6 GFP construct
tttaagcttggatcccaaggagatataacaatgctgctgctgctgctgctgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 12 - protein, 244 aa, Artificial Sequence; identical to SEQ ID NO: 8
  Leu(CTG)6 GFP construct

SEQ ID NO: 13 - DNA, 780 bp, Artificial Sequence
  Leu(CTT)6 GFP construct
tttaagcttggatcccaaggagatataacaatgcttcttcttcttcttcttagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 14 - protein, 244 aa, Artificial Sequence; identical to SEQ ID NO: 8
  Leu(CTT)6 GFP construct

SEQ ID NO: 15 - DNA, 780 bp, Artificial Sequence
  Leu(TTA)6 GFP construct
tttaagcttggatcccaaggagatataacaatgttattattattattattaagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 16 - protein, 244 aa, Artificial Sequence; identical to SEQ ID NO: 8
  Leu(TTA)6 GFP construct

SEQ ID NO: 17 - DNA, 780 bp, Artificial Sequence
  Leu(TTG)6 GFP construct
tttaagcttggatcccaaggagatataacaatgttgttgttgttgttgttgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 18 - protein, 244 aa, Artificial Sequence; identical to SEQ ID NO: 8
  Leu(TTG)6 GFP construct

SEQ ID NO: 19 - DNA, 780 bp, Artificial Sequence
  Lys(AAA)6 GFP construct
tttaagcttggatcccaaggagatataacaatgaaaaaaaaaaaaaaaaaaagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 20 - protein, 244 aa, Artificial Sequence
  Lys(AAA)6 GFP construct
MKKKKKKSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 21 - DNA, 780 bp, Artificial Sequence
  Lys(AAG)6 GFP construct
tttaagcttggatcccaaggagatataacaatgaagaagaagaagaagaagagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcctgcagaaa

SEQ ID NO: 22 - protein, 244 aa, Artificial Sequence; identical to SEQ ID NO: 20
  Lys(AAG)6 GFP construct

SEQ ID NO: 23 - DNA, 79 bp, Artificial Sequence
  3′ oligonucleotide primer
tttctgcaggaattcttatttgtatagttcatccatgccatgtgtaatcccagcagctgttacaaactcaagaaggacc

SEQ ID NO: 24 - DNA, 107 bp, Artificial Sequence
  5′ oligonucleotide primer incorporating His(CAC)6
tttaagcttggatcccaaggagatataacaatgcaccaccaccaccaccacagtaaaggagaagaacttttcactggagttgtcccaattcttgttgaattagatgg

SEQ ID NO: 25 - DNA, 107 bp, Artificial Sequence
  5′ oligonucleotide primer incoporating His(CAT)6
tttaagcttggatcccaaggagatataacaatgcatcatcatcatcatcatagtaaaggagaagaacttttcactggagttgtcccaattcttgttgaattagatgg

SEQ ID NO: 26 - DNA, 107 bp, Artificial Sequence
  5′ oligonucleotide primer incorporating Leu(CTA)6
tttaagcttggatcccaaggagatataacaatgctactactactactactaagtaaaggagaagaacttttcactggagttgtcccaattcttgttgaattagatgg

SEQ ID NO: 27 - DNA, 107 bp, Artificial Sequence
  5′ oligonucleotide primer incorporating Leu(CTC)6
tttaagcttggatcccaaggagatataacaatgctcctcctcctcctcctcagtaaaggagaagaacttttcactggagttgtcccaattcttgttgaattagatgg

SEQ ID NO: 28 - DNA, 107 bp, Artificial Sequence
  5′ oligonucleotide primer incorporating Leu(CTG)6
tttaagcttggatcccaaggagatataacaatgctgctgctgctgctgctgagtaaaggagaagaacttttcactggagttgtcccaattcttgttgaattagatgg

SEQ ID NO: 29 - DNA, 107 bp, Artificial Sequence
  oligonucleotide primer incorporating Leu(CTT)6
tttaagcttggatcccaaggagatataacaatgcttcttcttcttcttcttagtaaaggagaagaacttttcactggagttgtcccaattcttgttgaattagatgg

SEQ ID NO: 30 - DNA, 107 bp, Artificial Sequence
  5′ oligonucleotide primer incorporating Leu(TTA)6
tttaagcttggatcccaaggagatataacaatgttattattattattattaagtaaaggagaagaacttttcactggagttgtcccaattcttgttgaattagatgg

SEQ ID NO: 31 - DNA, 107 bp, Artificial Sequence
  oligonucleotide primer incorporating Leu(TTG)6
tttaagcttggatcccaaggagatataacaatgttgttgttgttgttgttgagtaaaggagaagaacttttcactggagttgtcccaattcttgttgaattagatgg

SEQ ID NO: 32 - DNA, 107 bp, Artificial Sequence
  5′ oligonucleotide primer incorporating Lys(AAA)6
tttaagcttggatcccaaggagatataacaatgaaaaaaaaaaaaaaaaaaagtaaaggagaagaacttttcactggagttgtcccaattcttgttgaattagatgg

SEQ ID NO: 33 - DNA, 107 bp, Artificial Sequence
  5′ oligonucleotide primer incorporating Lys(AAG)6
tttaagcttggatcccaaggagatataacaatgaagaagaagaagaagaagagtaaaggagaagaacttttcactggagttgtcccaattcttgttgaattagatgg

SEQ ID NO: 34 - DNA, 89 bp, Artificial Sequence
  5′ oligonucleotide primer control
tttaagcttggatcccaaggagatataacaatgagtaaaggagaagaacttttcactggagttgtcccaattcttgttgaattagatgg

SEQ ID NO: 35 - DNA, 730 bp, Artificial Sequence
  m-gfp5-minus ER targeting (5′) and retention signal (3′)
gcctcgagagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatgagagctccg

SEQ ID NO: 36 - protein, 237 aa, Artificial Sequence
  m-gfp5-minus ER targeting (5′) and retention signal (3′)
SKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 37 - DNA, 20 bp, Artificial Sequence
  35S Forward Primer
ccttcgcaagacccttcctc

SEQ ID NO: 38 - DNA, 21 bp, Artificial Sequence
  NOS Reverse Primer
tagtttgcgcgctatattttg